(19) United States
(12) Patent Application Publication
(10) Pub. No.: US 2016/0139141 A1
(43) Pub. Date: May 19, 2016

(54) IMAGING MASS CYTOMETRY USING MOLECULAR TAGGING

(71) Applicant: Fluidigm Canada Inc., Markham (CA)

(72) Inventor: Alexander V. Loboda, Thornhill (CA)

(21) Appl. No.: 14/940,951

(22) Filed: Nov. 13, 2015

Related U.S. Application Data
(60) Provisional application No. 62/079,448, filed on Nov. 13, 2014.

(51) Int. Cl. G01N 33/68

(52) U.S. Cl. CPC G01N 33/6848

(57) ABSTRACT

Methods of imaging a biological sample by mass cytometry using molecular tagging are disclosed.

### CROSS-REFERENCE TO RELATED APPLICATIONS

The present application claims priority to and the benefit of U.S. Provisional Patent Application No. 62/079,448 filed Nov. 13, 2014, the contents of which are incorporated herein by reference.

### FIELD

The present disclosure relates to methods and systems for imaging a biological sample by mass cytometry using molecular tagging.

### BACKGROUND

Mass cytometry is a popular tool for flow cytometry analysis of biological samples. In certain implementations, mass cytometry is based on affinity probing of antigens in biological cells using affinity probes having elemental tags. Tagged samples can then be analyzed by injecting material into an inductively coupled plasma (ICP) ion source where the elemental tags are atomized and ionized. The ionized cloud containing the elemental tags can be sampled into a mass spectrometer for analysis. CyTOF2 (Fluidigm Canada, Inc.) is a current commercial platform for mass cytometry. A benefit of using elemental tagging in mass cytometry is in the ability to simultaneously measure a large number of probes. For example, over 40 elemental probes can be analyzed in the ionized cloud from each sample.

Recently, the application of the mass cytometry has been extended to the field of immunohistochemistry-based imaging. This method is referred to as imaging mass cytometry (IMC). In IMC, a tissue is stained with affinity probes containing elemental tags. The spatial distribution of the elemental tags across the tissue is then analyzed using mass cytometry. For example, stained tissue can be subjected to laser ablation (LA) and then sampled into an ICP source for further analysis by mass spectrometry. In IMC, the quantitative distribution of target molecules can be determined indirectly by measuring the elemental tag attached to the affinity probe. Alternatively, the spatial distribution of the elemental tags can be determined using secondary ion mass spectrometry (SIMS).

At the same time as mass cytometry is developing, other techniques are actively being developed for imaging of biological samples. In imaging mass spectrometry (IMS), biological molecules are directly lifted intact from a tissue sample and the molecules ionized and detected as organic molecular ions using mass spectrometry. The spatial distribution of molecules of interest is determined by scanning across the sample. One of the advantages of IMS over optical imaging methods for determining the spatial distribution of molecules of interest is that it is not necessary to first stain the tissue prior to visualizing and analyzing the molecules. IMS methods, however, are limited in their ability to resolve complex molecules in the presence of many other organic molecules and are ineffective with poorly ionizing molecules.

In some proposed IMC approaches molecular tags are used in which the probe is cleaved from an affinity moiety during the sample desorption/ionization process or by collisions after the probe is ionized.

Improved methods of imaging target molecules in biological tissue are desired.

### SUMMARY

Methods provided by the present disclosure use molecular tagging in combination with IMS tools in which a reporter moieties are cleaved from respective affinity moieties prior to desorption and ionization.

In a first aspect, methods of imaging a biological sample by mass cytometry are provided, comprising: providing a biological sample; staining the biological sample with a molecular tag to provide a stained biological sample, wherein the molecular tag comprises an ionizable reporter moiety and an affinity moiety; releasing or partially releasing the ionizable reporter moiety from the affinity moiety on at least a portion of the stained biological sample; injecting the portion of the stained biological sample and the ionizable reporter moiety into a gas phase; and analyzing the ionizable reporter moiety.

Reference is now made in detail to certain embodiments of compounds, compositions, and methods. The disclosed embodiments are not intended to be limiting of the claims. To the contrary, the claims are intended to cover all alternatives, modifications, and equivalents.

### BRIEF DESCRIPTION OF THE DRAWINGS

FIG. 1 illustrates an exemplary method according to some embodiments of the disclosure.

### DETAILED DESCRIPTION

### Definitions

An affinity moiety refers to a chemical moiety capable of binding to or attaching to a specific molecular and/or chemical target. An affinity moiety is part of a molecular tag. When an affinity moiety is released or cleaved from a molecular tag, the affinity moiety is referred to as an affinity molecule.

An ionizable reporter moiety refers to a chemical moiety capable of being detected using mass spectrometry. An ionizable reporter moiety is part of a molecular tag. When an ionizable reporter moiety is separated from or cleaved from a molecular tag, the ionizable reporter moiety is referred to as an ionizable reporter molecule.

Antibodies refer to immunoglobulin glycoprotein molecules. Antibodies can be found in serum of animals. Antibodies may be made in mammals such as rabbits, mice, rats, goats, etc., and chicken. Procedures for immunization and elicitation of a high antibody production response in an animal are well known to those skilled in the art. Antibodies may also be made in cell cultures, for example by recombinant DNA methods. Antibodies may be used, for example, as whole molecules, half molecules known as Fab′ and Fab^{2′} fragments, or as monovalent antibodies (combining a light chain and a modified heavy chain).

### Methods

Methods provided by the present disclosure combine molecular tagging and mass cytometry methods for imaging of biological tissue.

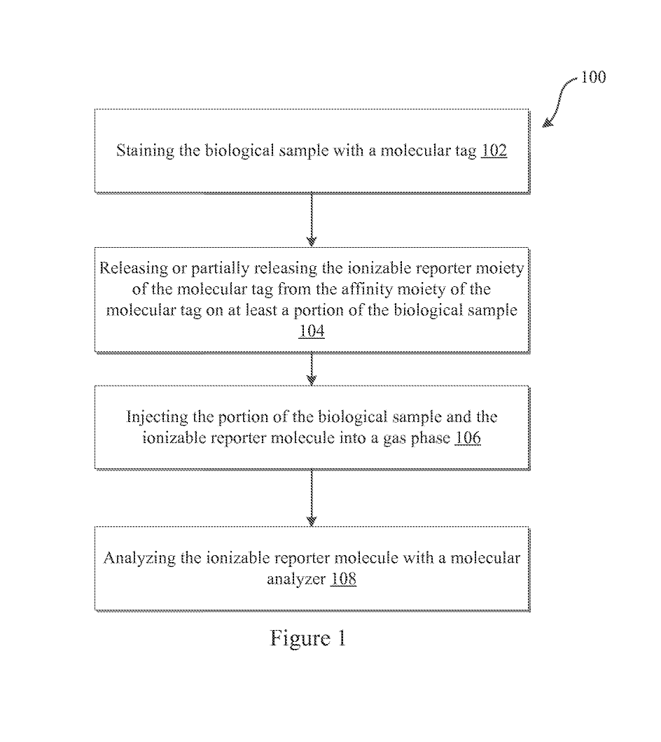

FIG. 1 illustrates an exemplary method 100 according to some embodiments of the disclosure. The exemplary method 100 includes staining a biological tissue of interest with a molecular tag 102. Molecular tags can contain an affinity moiety and an ionizable reporter moiety. An affinity moiety can be a moiety that binds to or attaches to a specific target molecule or target molecular site. An ionizable reporter moiety can be a moiety that can be preferentially ionized over other molecules in the tissue sample. The affinity moiety and the ionizable reporter moiety can be cleaved or partially cleaved or the ionizable reporter moiety may otherwise be released or partially released 104 from a portion of the biological tissue. Thereafter, the portion of the biological sample and/or the ionizable reporter molecule may be injected into the gas phase 106. Thereafter, the ionizable reporter molecule may be analyzed by a suitable molecular analyzer 108. Accordingly, in some embodiments, the ionizable reporter molecule may be at least partially released or cleaved prior to desorption from the tissue sample and/or before ionization. Cleaving can be accomplished, for example, chemically, photolytically, thermally, enzymatically, or by any other suitable methods. As in IMC, the tissue sample is imaged by scanning across the tissue sample and analyzing the distribution of the ionizable reporter moiety using mass cytometry.

Similar to fluorescent labeling fluorescent microscopy in which a fluorescent moiety is attached to an affinity moiety such as an antibody or other specific affinity molecule, a molecular tag contains both an affinity moiety and a reporter moiety that is readily ionizable and which can be analyzed using mass spectrometry.

Using a combination of molecular tags with each individual molecular tag having a unique affinity moiety and a unique reporter moiety a large number of simultaneous measurements can be made using imaging mass spectrometry. In certain embodiments, hundreds or thousands of unique ionizable reporter moieties can be simultaneously resolved using imaging mass spectrometry with molecular tagging.

### Biological Sample

A biological sample may be a liquid phase sample or a solid phase sample.

A biological sample can be any sample of a biological nature, or any sample suspected of comprising a biological samples. For example, a biological sample may include biological molecules, tissue, fluid, and cells of an animal, plant, fungus, or bacteria. A biological sample also includes molecules of viral origin. Examples include sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes. Another source of biological samples are viruses and cell cultures of animal, plant, bacteria, fungi where gene expression states can be manipulated to explore genomics and proteomics. Biological samples may also include solutions of purified biological molecules such as proteins, peptides, antibodies, DNA, RNA, aptamer, polysaccharides, lipids, etc.

In certain embodiments, a biological sample includes a tissue sample. The biological sample may be a thin section. A tissue section can be a thin section of biological tissue that may be frozen or paraffin-embedded and having a thickness from about 5 μm to about 20 μm.

A biological sample includes target molecules such as, for example, proteins, DNA, RNA, and other molecules present in a biological sample. The number and distribution of target molecules may reflect phenomena such as gene expression, protein expression, disease, or other property. A distribution of target molecules may include a distribution within a particular cell type, a distribution among different cell types, and/or a distribution within a particular tissue. Both the presence and/or the quantification of a particular target molecule may be of interest.

In certain embodiments, a biological sample can include cells captured on a substrate or particles of biological material captured on a substrate.

### Molecular Tagging

In certain embodiments, a molecular tag includes an affinity moiety and a reporter moiety.

An affinity moiety is selected to target a specific molecule and/or chemical site. An affinity moiety may bind to or chemically associate with a target. In certain embodiments, it is desirable that an affinity moiety remain bound to a target during sample processing such as during cleaving of the reporter moiety from the affinity moiety. In certain embodiments, it is desirable that an affinity moiety remain bound to a target during sample desorption and/or ionization of the reporter moiety. Sample desorption is understood to encompass a wide range of methods that allow for imaging of molecular composition in a particular location on the surface of a sample. These desorption methods and systems may range from laser desorption to liquid extraction; to desorption by secondary ions (such as SIMS). For example, liquid phase imaging may be used for sample desorption. Additionally, laser ablation and electrospray ionization may be used individually or in combination for sample desorption.

In certain embodiments, an ionizable reporter moiety can be selected to be preferentially ionizable compared to other molecules in a biological sample and/or compared to other parts of the molecular tag such as the affinity moiety and linker.

The affinity moiety and the reporter moiety are configured to be cleaved or partially cleaved prior to ionization.

In certain embodiments, an affinity moiety and an ionizable reporter moiety may be directly covalently bound, and in certain embodiments form a non-covalently bound complex with a target.

In certain embodiments, an affinity moiety and an ionizable reporter moiety may be covalently bound through a linker moiety. A linker moiety may provide a chemical structure to covalently bind an affinity and an ionizable reporter moiety. A linker moiety may also provide a chemical structure to facilitate cleaving the affinity moiety and the ionizable reporter moiety.

In certain embodiments, a linker moiety may be multidentate. For example, a multidentate linker may be used to bind multiple ionizable reporter moieties to a single affinity moiety. Alternatively, in certain embodiments, a multidentate linker may be used to bind multiple affinity moieties to a single ionizable reporter moieties, or multiple affinity moieties to multiple ionizable reporter moieties, where each of the multiple affinity and ionizable reporter moieties may be the same or different, or at least some of the multiple affinity moieties and/or the multiple ionizable reporter moieties may be the same or different.

In embodiments comprising multiple linker moieties, at least some of or each of the multiple linker moieties may be chemically different. The linker moieties may be different to accommodate different chemistries for binding an affinity moiety and an ionizable reporter moiety. Linker moieties may also differ in the ability or mechanism of cleaving the affinity moiety and the ionizable reporter moiety of a molecular tag. In terms of mechanism, some linker moieties may facilitate cleaving by different chemical, photochemical, or ionization mechanisms. Linker moieties may facilitate cleaving by similar mechanism but be responsive to different thresholds or conditions. For example, a linker moiety may facilitate cleaving at certain irradiation wavelengths and/or power densities.

In certain embodiments, a molecular tag includes a single affinity moiety and one or more ionizable reporter moieties. For example, in certain embodiments, a molecular tag may include one, two, three, four, or more ionizable reporter moieties. Use of multiple reporter moieties can serve to increase the sensitivity of a molecular tag.

In certain embodiments a molecular tag contains more than one ionizable reporter and in such embodiments each of the more than one ionizable reporter moiety may be the same, and in certain embodiments, at least one of the more than one ionizable reporter moieties is different. In certain embodiments, each of the more than one ionizable reporter moieties is different.

An ionizable reporter moiety may be cleaved from an affinity moiety before desorption and/or ionization of the ionizable reporter moiety. The moieties can be cleaved, for example, chemically, enzymatically, photolytically, thermally, or by any other suitable process. When cleaved from the affinity moiety, an ionizable reporter moiety remains localized on the tissue sample thereby preserving the information associated with the affinity moiety. In other words, following cleaving, the ionizable reporter moiety does not substantially migrate from the location of the affinity moiety.

Compared to biological molecules such as proteins, lipids, and oligonucleotides present in the sample, an ionizable reporter moiety represents a low molecular weight species that can be more readily released or desorbed from the tissue sample.

In certain embodiments, a molecular tag comprises a plurality of molecular tags. Although each of the molecular tags may be the same, for example, having the same affinity moiety, ionizable reporter moiety, and if present, linker moiety, a particular advantage of the disclosed method involves the use of molecular tags having different affinity moieties and ionizable reporter moieties.

In certain embodiments, a plurality of molecular tags may have a different affinity moiety and the same ionizable reporter moiety, but may have different linker groups. For example, the different linker groups may impart a different cleaving mechanism or for the same or similar cleaving mechanism can impart a different cleaving threshold or property. For example, although the plurality of molecular tags may be cleaved photolytically, the different linker moieties may impart the ability to cleave at different irradiation power thresholds and/or at different irradiation wavelengths. Such a method may allow the reuse of the same ionizable reporter moiety which may be read in a plurality of passes to provide spatial distribution of different target molecules.

In certain embodiments, a plurality of molecular tags comprises molecular tags having different affinity moieties and different ionizable reporter moieties, which may be cleaved by the same, similar, or by different mechanisms. The use of a plurality of molecular tags facilitates the ability to detect and/or quantify a plurality of molecular targets of a tissue sample. This greatly increases the ability of the disclosed methods to simultaneously measure multiple targets.

In certain embodiments, a molecular tag has the structure of Formula (1):

(A-X)ₙ—B (1)

wherein, A comprises an ionizable reporter moiety; X comprises a linker; B comprises an affinity moiety; and n is an integer of at least 1.

In certain embodiments, a molecular tag may be a DNA-based molecular tag in which the affinity moiety is a DNA-based aptamer, the linker is a DNA sequence specific for enzymatic cleavage and the ionizable reporter moiety is also a DNA sequence. To enhance the sensitivity of the linker and the ionizable reporter moiety can be replicated to provide multiple copies. This can provide a DNA-based molecular tag that is programmed for synthesis as necessary.

### Affinity Moiety

An affinity moiety may be any suitable moiety configured to bind or attach to a specific target molecule, a specific chemical site, or a combination thereof.

In certain embodiments, an affinity moiety may be an antibody, lectin, oligonucleotides, aptamer, or other chemical species capable of binding to a particular biological molecule.

In certain embodiments, the affinity moiety binds to or associates with the target species to an extent that it is not dissociated during cleavage from the ionizable reporter moiety. In certain embodiments, the affinity moiety may be separated from the target species during cleavage from the ionizable reporter moiety.

Target species refers to a molecule of interest that is capable of specifically binding to an affinity moiety. Examples of target molecules include nucleic acids, in particular mRNA molecules, peptides, proteins, in particular receptors and ligands, antibodies, antigens, haptens, and organic compounds. The tandem target/binding molecules may display any chemical structure capable of generating a specific hybridization in a tissue section. Examples of tandem target/binding molecules include including nucleic acids/nucleic acids, nucleic acids/peptides, nucleic acids/proteins, nucleic acids/antibodies, peptides/peptides, peptides/proteins, peptides/antibodies, proteins/proteins (in particular ligands/receptors), proteins/sugars, antigens/antibodies, haptens/antibodies, organic compounds/receptor

In the case of peptides and proteins, any suitable peptidic ligand/peptidic receptor tandem molecules may represent a target. Such peptidic ligand/peptidic receptor tandem molecules include peptidic antigens/antibodies or antibody fragments, as well as any hormone/hormone receptor, cytokine/cytokine receptor tandem, chemokine/chemokine receptor, aptamer/peptide, aptamer/protein. Membrane sugars that are implicated in cell migration and their proteic receptors are also possible targets.

In certain embodiments, a target molecule may be an antigen such as nucleic acids, haptens, peptides or proteins and their specific antibodies are included in the scope of a molecular tag.

Organic compounds may also be mapped using methods provided by the present disclosure. For example, the in vivo distribution of administered organic drugs may be monitored using the disclosed methods.

### Reporter Moiety

In certain embodiments, a reporter moiety is readily ionizable under typical experimental conditions and in certain embodiments, is preferentially ionizable from other molecules present in the sample.

In certain embodiments, in addition to be readily ionizable an ionizable reporter moiety may be configured to be easily lifted from the biological sample for analysis using mass spectrometry. For example, an ionizable reporter moiety, when separated from the affinity moiety may exhibit a low vapor pressure, may have a low molecular mass, and/or may be easily cleaved or separated from the affinity moiety. Accordingly, in some embodiments, an ionizable reporter moiety may be configured to facilitate transfer from the specimen into the gas phase when desorption or ablation probes are used. In additional embodiments, the ionizable reporter moiety may be configured to facilitate transfer into a liquid stream for the methods which rely on liquid extraction for imaging mass spectrometry.

An ionizable reporter moiety may be characterized by a number of attributes such as, for example, ionization efficiency, mass to charge ratio, mass, vapor pressure, structure, or a combination of any of the foregoing. In some embodiments, reporter moiety mass may preferably be in the 50-500 amu range or in 100-3000 amu range or in 500-10,000 amu range; or in 3-300 kamu range. For charge, in some embodiments, the reporter ion may carry a single elemental charge; or 2 charges; or several charges in the range of 3-20; or several charges in the range of 10-100 or several charges in the range of 30-3000. It may also occur that a single reported moiety will be recorded as several mass/charge peaks that vary in the numbers of charges present on the reporter moiety.

Based on a subset of these and/or other attributes a collection of ionizable reporter moieties can be provided.

For example, in certain embodiments, a plurality of ionizable reporter moieties may be distinguished by molecular mass. In certain embodiments, an ionizable reporter moiety is characterized by a mass from 200 amu to 1,000 amu, from 200 amu to 800 amu, from 200 amu to 600 am, and in certain embodiments, from 200 amu to 400 amu.

In other embodiments, each of a plurality of ionizable reporter moieties may have the same mass but be characterized by a different structure, composition, or a combination thereof, which difference is resolvable using mass spectrometry, such as using tandem mass spectrometry (MS-MS), or ion mobility separation methods. The difference in structure and/or composition can be made such that fragment ions can be distinguished using, for example, MS-MS. Unfragmented, i.e., non-ionized, reporter moieties will be characterized by the same mass, i.e., be isobaric. Thus, the unfragmented ions can pass through a narrow mass filter, which can also eliminate potentially contaminating molecular species having other masses. The isobaric reporter moieties can then be separated, using MS-MS techniques. The use of isobaric ionizable reporter moieties can be particularly attractive for imaging mass cytometry with molecular tagging as a way to separate the reporter molecules from other molecular species. The samples may be introduced by desorption, laser ablation, liquid sampling, or the like.

Isobaric tags are described, for example, in U.S. Application Publication No. 2013/0078728 and in U.S. Application Publication No. 2014/0273252.

An ionizable reporter moiety may be selected to have a high ionization efficiency compared to other molecules in a sample. For example, the electrospray ionization efficiency of small organic compound can range over six orders of magnitude. Oss et al., “Electrospray ionization efficiency scale of organic compounds,” Anal. Chem. 82(7), 2010, 2865-2872; Nguyen et al., “An approach toward quantification of organic compounds in complex environmental samples using high-resolution electrospray ionization mass spectrometry,” Anal. Methods 2013, 5, 72-80; Kruve et al., “Negative electrospray ionization via deprotonation: Predicting the ionization efficiency,” Anal. Chem 2014, 86, 4822-4830.

In general, compounds that are more basic, larger molecular volumes, increasing number of alkyl chains, molecular size, generally exhibit increased ionization efficiency for positive ions. The ionization efficiency for negative ions may be increased for acidic molecules.

Ionizable reporter moieties may be selected to be readily ionizable. Many parameters can affect ionization efficiencies including polarizability, gas-phase basicity (GB), related to proton affinity (PA) by an entropic term −TΔS°), sodium affinity, and surface activities; and these properties are affected by both the molecular size and the structure of the molecule.

For homologous series of compounds, GB and average polarizability of compounds are proportional to the molecular size.

GB is also intrinsically related to structural characteristics such as the ionization site or degree of unsaturation. Specifically, because the additional pi-electrons offer resonance stabilization of the positive charge, GB increases with the degree of unsaturation in molecules when ionization occurs on carbon atoms, such as for aliphatic hydrocarbons, carbonyls, and cyclic ethers. However, when ionization occurs on more basic atoms such as N, S, or O, GB decreases with the degree of unsaturation due to the conversion from the sp3 hybridization state to the sp2 state of the basic atoms, e.g., going from an amine to an enamine. Because the dependence of the ionization efficiency on structural properties, such as the degree of unsaturation, may vary by compound class, the molecular size alone is not directly correlated with the ionization efficiency.

In certain embodiments, an ionizable reporter moiety comprises a mass tag that represents a structural isomer, a conformer and/or chiral compound. These mass tags may be separated from others using ion mobility/mass spectrometry separation methods.

### Staining

Biological samples can be prepared for imaging by staining with a molecular tag. Staining can be accomplished using methods similar to those known in the art for staining biological samples with fluorescent affinity labels. A composition for staining may include a plurality of different molecular tags. Staining protocols are known in the art and can be selected based on the particular affinity moieties contained in the staining composition.

### Molecular Tag Separation

After a biological sample is stained with a molecular tag, the ionizable reporter moiety can be released or separated from the affinity moiety using any suitable methods. It is desirable that the released ionizable reporter moiety remain spatially associated with the affinity moiety to preserve the quantitative and positional information accessed by the affinity moiety.

In certain embodiments, an ionizable reporter moiety may be released chemically, enzymatically, photolytically, thermally, or other suitable methods. In chemical cleaving methods, a solution containing reactant, catalyst, pH buffer or other chemical may be applied to a surface of a stained sample to release or cleave the ionizable reporter moiety. The solution may be left in place to minimize diffusion of the released ionizable reporter moiety. In other methods, the stained biological sample may be irradiated with a suitable radiation source to photolytically cleave the ionizable reporter moiety. In certain embodiments, prior to irradiation, a sample may be treated with a photosensitizing agent such as a free radical generator to facilitate the photolytically induced reaction. In thermal methods, heat may be applied to a sample.

In certain embodiments, releasing includes partially releasing the ionizable reporter moiety from the associated affinity moiety. In certain embodiments, partially releasing refers to changing the bonding relationship between the ionizable reporter moiety and the affinity moiety such that full cleavage or separation during desorption and/or ionization can be facilitated. For example, an ionizable reporter moiety may be covalently bound to an affinity moiety. The molecular tag may be treated such that the covalent bond is weakened or changed to a non-covalent bond. In certain embodiments, the chemistry of the covalent bond may be altered such that the ionizable reporter moiety is rendered easier to release during ablation and/or ionization. Benefits of this approach include the ability of the ionizable reporter moiety to remain localized at the associated target site.

In certain embodiments, the ionizable reporter moiety is not released by ionization or during ionization. The methods provided by the present disclosure are distinguished from those in which an ionizable reporter moiety is cleaved from an affinity moiety during ablation and/or ionization such as MALDI. In the methods provided in the present disclosure, the cleavage of the ionizable reporter moiety is separate from the ablation/desorption and ionization processes. The disclosed methods facilitate the use of a larger range of cleavage mechanisms that can be precisely tailored for particular molecular tags.

Molecular tag cleavage can be performed across the surface of a biological sample of interest, over a portion of a biological sample, or locally to conform to a particular area being sampled by a mass spectrometer. Localized cleavage is more suitable to methods where the ionizable reporter molecule is cleaved photolytically or thermally where, for example, a laser can be used to effect cleavage before or at the same time an ionizable reporter moiety is ablated, desorbed, or otherwise released from the sample.

### Gas Phase

Following cleavage of the molecular tag, the ionizable reporter molecule can be injected into the gas phase and ionized for analysis by mass spectrometry. The reporter molecules may be moved away from the solid state at their location on the biological sample being interrogated. In some cases, the reporter molecule can enter a gas flow. Optionally, it can enter a stagnant gas media. In further embodiments, it may enter a vacuum. This process may involve ablation or desorption or combinations thereof. For example, in desorption electrospray ionization, the desorption may be carried out without laser. In additional examples, SIMS may be used where desorption is provided by ion impact rather than ablation. There are many desorption/ionization methods developed for IMS. Examples of suitable desorption/ionization including, for example, ELDI, LAESI, MALDESI, DESI, DAPPI, DART, LMJ-SSP, LESA, SIMS, liquid microjunction surface sampling, laser ablation liquid microjunction sampling (Ovchinnikova et al., “Laser ablation sampling of materials directly into the formed liquid microjunction of a continuous flow surface sampling probe/electrospray ionization emitter for mass spectral analysis and imaging,” Anal. Chem. 2013, 85, 10211-10217), and nanospray desorption electrospray ionization (Laskin et al., “Tissue imaging using nanospray desorption electrospray ionization mass spectrometry,” Anal. Chem 2012, 84, 141-148).

Ionizable reporter molecules can be desorbed or separated from the sample by any suitable method such as by irradiating a portion of a biological sample with photons or high-energy particles such as in, for example, SIMS, or by introducing each portion into a liquid phase with a subsequent ionization into a gas phase, such as done, for example, in microjunction sampling methods.

In certain embodiments, gas phase samples may be produced using femtosecond laser irradiation. Femtosecond laser pulses can provide, for example, 1 μm resolution or less, and ablation can be accomplished using only a few nanojoules of energy.

The ionizable reporter molecules, which have been cleaved or separated from the respective affinity moiety, can be ionized during desorption from biological sample or within a mass spectrometer during a subsequent ionization step.

### Mass Analysis

Ionized reporter molecules and/or fragments thereof may be analyzed using any suitable mass spectrometry method. In certain embodiments, it is desirable that the reporter molecules be determined qualitatively and in certain embodiments, quantitatively.

In certain embodiments, such as when isobaric reporter moieties are employed, tandem mass spectrometer analysis can be appropriate. Tandem mass spectrometers are mass spectrometers that are capable of performing multiple mass analysis steps and changing the composition of ions, for example, via fragmentation, prior to one or more of the subsequent mass analysis steps. A mass spectrometer that is capable of performing two mass analysis steps is referred to as a MS-MS mass spectrometer and a tandem mass spectrometer capable of performing n mass analysis steps is referred to as an MS' mass spectrometer. Tandem mass spectrometers can be characterized as being either tandem-in-space or tandem-in-time. Tandem-in-space mass spectrometers have physically separated mass analyzers. Tandem-in-time mass spectrometers use the same mass analyzer(s) over and over again to perform sequentially all steps of selection and readout. A wide variety of tandem mass spectrometers with various types of mass analyzer sections are known in the art. The mass analyzer sections in the tandem mass spectrometers can be the same or can be different types of mass analyzers. For example, there are tandem mass spectrometers with quadrupole-quadrupole, magnetic sector-quadrupole, quadrupole-linear-ion-trap, and quadrupole-time-of-flight mass analyzers.

Examples of mass spectrometers useful in methods provided by the present disclosure include tandem-in-time mass spectrometers, such as RF-ion trap (linear and 3-D), ion cyclotron resonance (which is also known as Penning trap and Fourier Transform Mass Spectrometer-FTMS), and hybrid mass spectrometers, such as quadrupole-linear-ion trap or quadrupole-FTMS. Accordingly, a mass spectrometer instrument may receive a portion of the reporter ions and then may correlate these ions to a particular location on the specimen to produce imaging mass spectrometry data. Thus, any mass analyzer may be used with embodiments described herein. To enable imaging mass cytometry on these mass analyzer machines, the instrument may be configured for imaging mass spectrometry of samples “stained” with affinity reagents containing reporter molecules.

In certain embodiments, ion mobility mass spectrometry methods can be employed to resolve reporter molecules.

Isomers of the primary structure (“structural isomers”) and isomers of the secondary or tertiary structure (“conformational isomers”) possess different geometrical shapes but exactly the same mass. Mass spectrometry is therefore unable to detect that they are different. One of the most efficient methods of recognizing and distinguishing such isomers is to separate them by virtue of their ion mobility. In certain embodiments, a cell for measuring the ion mobility contains an inert gas (such as helium or nitrogen). The ions of the substance under investigation are usually pulled through the stationary gas by means of an electric field. The large number of collisions with the gas molecules leads to a constant drift velocity v_{d} for every ionic species which is proportional to the electric field strength E: v_{d}=M×E. The proportionality factor M is called the “ion mobility”. The ion mobility M is a function of the temperature, gas pressure, type of gas, ionic charge and, in particular, the collision cross-section. Isomeric ions of the same mass but different collision cross-sections possess different ion mobilities. Isomers with the smallest geometry possess the largest mobility M and therefore the largest drift velocity v_{d} through the gas. Protein ions which are unfolded undergo more collisions than tightly folded proteins. Unfolded protein ions therefore arrive at the end of the cell later than folded ions of the same mass.

A variety of information can be obtained from measurements of the ion mobility M. Measurements of the relative ion mobility can be used to investigate conformational changes or merely to discover the existence of different isomeric structures in a mixture. Ions with the same mass-to-charge ratio m/z but different conformation can be separated from each other relatively easily. It is even possible to calculate the absolute collision cross-sections from well reproduced measurements with helium as the gas. Specific folding forms can be confirmed in turn from the accurate collision cross-sections.

Knowledge of the mobility of ions has become more and more important in chemical and biological research, and devices for measuring ion mobility have therefore been incorporated in mass spectrometers in order to combine measurements of the mass-to-charge ratio of ions with measurement of collision cross-sections.

Examples of ion mobility mass spectrometers are disclosed, for example, in U.S. Pat. No. 6,744,043 B2, U.S. Pat. No. 5,847,386, U.S. Application Publication No. 2010/0193678A1, U.S. Application Publication No. 2009/0189070, U.S. Application Publication No. 2011/0121171A1, U.S. Application Publication No. 2014/0042315, and in U.S. Application Publication No. 2014/0145076.

### Imaging Mass Cytometry

Biological samples such as tissue cross-sections can be imaged by scanning an ablation/desorption probe across a surface of the sample. As described above, many ablation/desorption methods may be utilized with embodiments described herein. For example, in some embodiments, a hot jet or even a plasma may be used to desorption/ablation. The ionizable reporter compounds are analyzed quantitatively and/or qualitatively and the results combined to generate a map or multiple maps of the biological sample. The map or maps can be two-dimensional representations of the target molecules across a biological sample. Further, it should be understood that three-dimensional profiles may be provided by embodiments of the present invention. For instance, a stack of two-dimensional images may be recorded of a specimen to reconstruct a three-dimensional profile. Optionally, a true three dimensional scanning may be provided that consistently removes layer by layer of the biological sample in order to provide a three-dimensional profile showing the target molecule distribution throughout the volume/space. In certain embodiments, an ablation/desorption probe is moved successively across the sample and data obtained for individual spots. The spots can have dimensions, for example, diameters from 0.10 μm to 200 μm depending on the method used.

Image reconstruction can be performed using any suitable image reconstruction software and techniques known in the art.

Using methods provided by the present disclosure several distinct target molecules can be mapped simultaneously. One of the advantages of using molecular tags is that multiple targets can be determined simultaneously. Using tag molecules with widely dispersed molecular weights, it is thus possible using any above described method according to the invention to map simultaneously the expression of many distinct target molecules in the same tissue section. Using the molecular tags and methods disclosed herein it can be possible to analyze anywhere from a single target molecule to several thousand target molecules simultaneously.

### Uses

By separating the function of molecular/chemical targeting and reporting a molecular tag can facilitate the study of molecules/sites that might otherwise be difficult to detect using conventional IMS. For example, a target site may consist of molecules that do not ionize under IMS conditions, that are not resolvable using IMS, or that are difficult to release from a biological sample. The latter situation can arise with large biopolymers.

Imaging mass cytometry using molecular tagging is also expected to exhibit certain advantages compared to elemental tagging. The ion transmission in elemental tagging imaging mass cytometry is relatively low. In contrast, in some mass spectrometer configurations, molecular transmission efficiencies can be as high as from about 10% to about 50%. As a result, the ability to detect affinity targets with imaging mass cytometry using molecular tagging will be greatly enhanced.

### Combination Analysis

Imaging mass cytometry using molecular tagging may be combined with other tissue imaging methods. For example, images derived from molecular tags may be combined with optical images and/or images obtained from fluorescent labels or isotopic labels of the same tissue sample.

### Apparatus

Embodiments provided by the present disclosure further include apparatus for implementing and employing methods provided by the present disclosure.

In certain embodiments, apparatus includes stages, imaging systems, vaporization apparatus, ionizers, and mass spectrometers adapted for use in imaging mass cytometry using molecular tags. Known apparatus may be adapted to optimize the detection, resolution, and characterization of the particular ionizable reporter moieties associated with the molecular tags used to stain a particular sample.

Finally, it should be noted that there are alternative ways of implementing the embodiments disclosed herein. Accordingly, the present embodiments are to be considered as illustrative and not restrictive. Furthermore, the claims are not to be limited to the details given herein, and are entitled their full scope and equivalents thereof.

What is claimed is:

1. A method of imaging a biological sample by mass cytometry, comprising:
providing a biological sample;
staining the biological sample with a molecular tag to provide a stained biological sample, wherein the molecular tag comprises an ionizable reporter moiety and an affinity moiety;
releasing or partially releasing the ionizable reporter moiety from the affinity moiety on at least a portion of the stained biological sample to provide an ionizable reporter molecule;
injecting the portion of the stained biological sample and the ionizable reporter molecule into a gas phase; and
analyzing the ionizable reporter molecule.

2. The method of claim 1, wherein the injecting step is followed by ionizing the ionizable reporter molecule.

3. The method of claim 1, wherein the biological sample comprises a tissue.

4. The method of claim 1, wherein analyzing comprises using an imaging mass spectrometry apparatus.

5. The method of claim 1, wherein the affinity moiety comprises an antibody.

6. The method of claim 1, wherein the ionizable reporter moiety is configured to be cleaved from the molecular tag.

7. The method of claim 1, wherein the ionizable reporter moiety is configured to be chemically cleaved from the molecular tag.

8. The method of claim 1, wherein the ionizable reporter moiety is configured to be photolytically cleaved from the molecular tag.

9. The method of claim 1, wherein the molecular tag comprises more than one ionizable reporter moiety.

10. The method of claim 1, wherein the molecular tag comprises more than one ionizable reporter moiety bonded to the affinity moiety.

11. The method of claim 1, wherein the ionizable reporter moiety is characterized by a mass from 200 amu to 1,000 amu.

12. The method of claim 1, wherein,
the ionizable reporter moiety is characterized by a mass and a structure; and
the ionizable reporter moiety is resolvable, using mass spectrometry, from another ionizable reporter moiety characterized by the same mass and a different structure.

13. The method of claim 1, wherein the molecular tag comprises a plurality of molecular tags, wherein the plurality of molecular tags comprise:
a first molecular tag comprising a first ionizable reporter moiety and a first affinity moiety; and
a second molecular tag comprising a second ionizable reporter moiety and a second affinity moiety.

14. The method of claim 13, wherein,
the first ionizable reporter moiety and the second ionizable reporter moiety are different; and
the first affinity moiety and the second affinity moiety are different.

15. The method of claim 13, wherein the first ionizable reporter moiety and the second ionizable reporter moiety are characterized by a different attribute selected from a mass, a structure, a chemical composition, and a combination of any of the foregoing.

16. The method of claim 13, wherein the first ionizable reporter moiety and the second ionizable reporter moiety are configured to be resolved by mass spectrometry, tandem mass spectrometry, ion mobility mass spectrometry, and a combination of any of the foregoing.

17. The method of claim 13, wherein the first ionizable reporter moiety and the second ionizable reporter moiety are characterized by the same mass and an attribute selected from a different structure, a different chemical composition, and a combination thereof.

18. The method of claim 13, wherein the first ionizable reporter moiety and the second ionizable reporter moiety are characterized by the same mass.

19. The method of claim 1, wherein injecting into the gas phase comprises laser ablating.

20. The method of claim 1, wherein injecting the portion of the stained biological sample and the ionizable reporter molecule comprises scanning an ablation or desorption probe across a surface of the portion of the sample.

21. The method of claim 20, wherein scanning the ablation or desorption probe across the surface of the portion of the sample comprises scanning the probe successively across an area; and wherein analyzing the ionizable reporter moiety comprises generating a map of the biological sample.

22. The method of claim 21, wherein the map shows a spatial distribution of the ionizable reporter molecule.

23. The method of claim 1, wherein releasing or partially releasing the ionizable reporter moiety from the affinity moiety comprises cleaving or partially cleaving the ionizable reporter moiety from the affinity moiety.

24. The method of claim 1, wherein releasing or partially releasing the ionizable reporter moiety comprises changing a bonding relationship between the ionizable reporter moiety and the affinity moiety.

25. A method of qualitatively or quantitatively analyzing a spatial distribution of a target molecule in a biological sample, the method comprising:
staining the biological sample with a molecular tag to provide a stained biological sample, wherein the molecular tag comprises an ionizable reporter moiety and an affinity moiety that is specific for the target molecule;
changing a bonding relationship between the ionizable reporter moiety and the affinity moiety on at least a portion of the stained biological sample;
after changing the bonding relationship between the ionizable reporter moiety and the affinity moiety, scanning a desorption probe or ablation probe across a surface of the portion of the sample to inject an ionizable reporter molecule into a gas phase; and
analyzing the ionizable reporter molecule to provide the spatial distribution of the target molecule in the biological sample.
